SEQUENCE LISTING (6 sequences)

SEQ ID NO: 1 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 2 - protein, 202 aa, Homo sapiens
NKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 3 - DNA, 663 bp, Homo sapiens
atgctcaggctgctcttggctctcaacttattcccttcaattcaagtaacaggaaacaagattttggtgaagcagtcgcccatgcttgtagcgtacgacaatgcggtcaaccttagctgcaagtattcctacaatctcttctcaagggagttccgggcatcccttcacaaaggactggatagtgctgtggaagtctgtgttgtatatgggaattactcccagcagcttcaggtttactcaaaaacggggttcaactgtgatgggaaattgggcaatgaatcagtgacattctacctccagaatttgtatgttaaccaaacagatatttacttctgcaaaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcctga

SEQ ID NO: 4 - protein, 27 aa, Homo sapiens
FWVLVVVGGVLACYSLLVTVAFIIFWV

SEQ ID NO: 5 - protein, 144 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSP

SEQ ID NO: 6 - protein, 126 aa, Homo sapiens
NKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSP

(19) United States
(12) Patent Application Publication
(10) Pub. No.: US 2023/0035730 A1
(43) Pub. Date: Feb. 2, 2023

(54) SOLUBLE CD28 LEVELS DURING IMMUNOTHERAPY

(71) Applicant: BIOND BIOLOGICS LTD., Misgav (IL)

(72) Inventors: Motti HAKIM, Kibbutz Gazit (IL); Anna FRIDMAN-DROR, Kibbutz Dalia (IL); Orit SHILOVIZKY, Haifa (IL); Dror ALISHEKEVITZ, Kiryat Tivon (IL); Ilana MANDEL, Manof (IL); Tehila BEN-MOSHE, Tel Aviv (IL); Yair SAPIR, Manof (IL); Avidor SHULMAN, Rakefet (IL)

(21) Appl. No.: 17/781,966

(22) PCT Filed: Dec. 2, 2020
(86) PCT No.: PCT/IL2020/051244

§ 371 Date: Jun. 2, 2022

Related U.S. Application Data
(60) Provisional application No. 62/942,276, filed on Dec. 2, 2019.

(51) Int. Cl. G01N 33/574

(52) U.S. Cl. CPC G01N 33/57488; G01N 2333/70521; G01N 2800/54

(57) ABSTRACT

Methods of diagnosing and predicting cancer relapse comprising measuring soluble CD28 levels in a subject, wherein an increase in soluble CD28 is indicative of cancer relapse or imminent cancer relapse, are provided. Methods of determining response to PD-1/PD-L1 based immunotherapy in a subject in need thereof, comprising measuring sCD28 levels in a subject undergoing PD-1/PD-L1 based immunotherapy at at least two time points wherein a decrease indicates response and an increase indicates lack of response, are also provided.

### CROSS REFERENCE TO RELATED APPLICATIONS

This application claims the benefit of priority of U.S. Provisional Patent Application No. 62/942,276, filed Dec. 2, 2019, the contents of which are all incorporated herein by reference in their entirety.

### FIELD OF INVENTION

The present invention is in the field of immune regulation and immunotherapy.

### BACKGROUND OF THE INVENTION

It is known that some co-stimulatory molecules have several physiological forms. Alongside membrane-bound forms, soluble forms have been described that are expressed in naive immune cells, increasing the complexity of T cell biology. The soluble form of CD28 (sCD28) has been implicated in inhibiting an immune response to cancer. Further, sCD28 has been demonstrated to suppress the effectiveness of PD-1 and PD-L1 based immunotherapies. It has been hypothesized that sCD28's presence may be a contributing factor to the heterogeneity of response to immunotherapy.

However, the status of sCD28 during the course of immunotherapy and its possible role in relapse has not been investigated. Further, the role sCD28 levels may have in patients becoming refractory to immunotherapeutic treatment is unknown.

### SUMMARY OF THE INVENTION

The present invention provides methods of diagnosing cancer relapse in a subject and of predicting imminent cancer relapse in a subject. The methods comprise measuring soluble CD28 (sCD28) levels in the subject, wherein an increase in sCD28 levels indicates the subject is in relapse or that relapse is imminent. Methods of determining response to PD-1/PD-L1 based immunotherapy in a subject in need thereof, comprising measuring sCD28 levels in a subject undergoing PD-1/PD-L1 based immunotherapy at at least two time points wherein a decrease indicates response and an increase indicates lack of response, are also provided.

According to a first aspect, there is provided a method of determining response to PD-1/PD-L1 based immunotherapy in a subject suffering from cancer, the method comprising measuring sCD28 levels in the subject at at least two time points wherein at least one of those time points is after initiation of the PD-1/PD-L1 based immunotherapy, wherein an increase in sCD28 levels from a first time point to a second time point after initiation of the PD-1/PD-L1 based immunotherapy indicates the subject is not a responder to the immunotherapy and a decrease or no change in sCD28 levels from the first time point to the second time point after initiation of the PD-1/PD-L1 based immunotherapy indicates the subject is a responder to the immunotherapy, thereby determining response to PD-1/PD-L1 based immunotherapy in a subject.

According to some embodiments, the first time point is before or at the time of initiation of the immunotherapy.

According to some embodiments, the first time point is after initiation of the immunotherapy.

According to some embodiments, the second time point is at least 6 weeks after the first time point.

According to some embodiments, the second time point is at most 7 weeks after the first time point.

According to some embodiments,
a. the first time point is at or before initiation of the immunotherapy and the second time point is 7 weeks or 13 weeks after initiation of the immunotherapy; or
b. the first time point is at 7 weeks after initiation of the immunotherapy and the second time point is 13 weeks after initiation of the immunotherapy.

According to some embodiments, a decrease in sCD28 levels from the first time point to the second time point after initiation of the PD-1/PD-L1 based immunotherapy indicates the subject is a responder to the immunotherapy.

According to some embodiments, a decrease is a decrease of at least 1 ng/ml sCD28.

According to another aspect, there is provided a method of diagnosing cancer relapse in a subject in need thereof, the method comprising measuring soluble CD28 (sCD28) levels in the subject, wherein an increase in sCD28 levels in the subject indicates cancer relapse, thereby diagnosing cancer relapse in the subject.

According to some embodiments, the method of the invention further comprises at least one of:
a. discontinuing the immunotherapy to a subject that is not a responder;
b. administering a different immunotherapy to a subject that is a non-responder;
c. continuing to administer said PD-1/PD-L1 based immunotherapy to a subject that is a responder; and
d. increasing a dose of the PD-1/PD-L1 based immunotherapy administered to a subject that is a responder.

According to another aspect, there is provided a method of predicting cancer relapse in a subject in need thereof, the method comprising measuring soluble CD28 (sCD28) levels in the subject, wherein an increase in sCD28 levels in the subject indicates an imminent cancer relapse, thereby predicting cancer relapse in the subject.

According to some embodiments, the subject suffers from cancer and the cancer is in remission.

According to some embodiments, the subject previously had cancer and is currently cancer free.

According to some embodiments, the subject has undergone or is undergoing immunotherapy.

According to some embodiments, the immunotherapy is PD-1, PD-L1 and/or CD80 based immunotherapy.

According to some embodiments, the increase is as compared to sCD28 levels in the subject before relapse.

According to some embodiments, the increase is as compared to a predetermined threshold.

According to some embodiments, the increase is an increase of at least 50%.

According to some embodiments, the increase is an increase to at least 6 ng/mL sCD28.

According to some embodiments, the increase is an increase of at least 1 ng/ml sCD28.

According to some embodiments, the measuring comprises obtaining a sample from the subject and measuring sCD28 levels in the sample.

According to some embodiments, the sample is a blood sample.

According to some embodiments, the measuring comprises measuring at a plurality of time points, wherein at least one time point is at a time when the subject is known to be in remission or cancer free, and wherein at least one time point is at a time when the subject is at risk of relapse.

According to some embodiments, remission comprises a partial response and a complete response to a therapy.

According to some embodiments, at the time point when the subject is known to be in remission or cancer free sCD28 levels are below 5 ng/mL.

According to some embodiments, the subject before performance of the method has blood sCD28 levels below 6 ng/mL.

According to some embodiments, the cancer is selected from skin cancer, urothelial cancer, lung cancer, and renal cancer.

According to some embodiments, the cancer is selected from melanoma, and urothelial carcinoma.

According to some embodiments, imminent is within the next 20 weeks.

According to some embodiments, the method of the invention further comprises administering another immunotherapy to a subject diagnosed with or predicted for cancer relapse.

According to some embodiments, the method of the invention further comprises administering another immunotherapy to a subject determined to be a non-responder to said immunotherapy.

According to some embodiments, the immunotherapy is selected from:
a. a checkpoint inhibitor;
b. a chimeric antigen receptor (CAR) based therapy; and
c. a cancer vaccine.

According to some embodiments, the checkpoint inhibitor is a PD-1 and/or PD-L1 based immunotherapy.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1. Survival chart of melanoma patients undergoing anti-PD1 therapy. Patients with a low level of sCD28 before initiation of the therapy had a longer mean survival than patients with high levels of sCD28.

FIGS. 2A-2D. (2A) Line graphs of the levels of sCD28 in 17 patients (9 responders and 8 non-responders) over the course of several weeks of immunotherapy. (2B-2C) Bar graphs of mean sCD28 levels in (2B) non-responders and (2C) responders to immunotherapy at three time points. (2D) Bar graph of absolute changes in sCD28 expression from the initial reading before initiation of immunotherapy to two time points after initiation. * is a Pval=0.06, ** is a Pval<0.05 according to the Signed Rank test.

FIG. 3 Line graphs of sCD28 levels from two patients that responded to immunotherapy and whose cancer then relapsed. CR is complete response; PR is partial response; PD is progressive disease; and SD is stable disease.

### DETAILED DESCRIPTION OF THE INVENTION

The present invention, in some embodiments, provides methods of diagnosing and predicting cancer relapse in a subject that had or still has cancer, comprising measuring soluble CD28 (sCD28 levels) in the subject, wherein an increase in sCD28 is indicative of cancer relapse or imminent cancer relapse. The invention further provides methods of determining response to PD-1/PD-L1 based immunotherapies, by measuring changes in sCD28 levels during the course of treatment with the immunotherapy. The invention is based on the surprising finding that in subjects that had been responding to immunotherapy a spike in sCD28 levels acts as a marker of relapse that has started or is about to start. It is further based on the surprising finding that sCD28 levels increase in subjects that are not responding to immunotherapy and do not change or decrease in subjects that are responding.

By a first aspect, there is provided a method of diagnosing cancer relapse in a subject in need thereof, the method comprising measuring soluble CD28 (sCD28) levels in the subject, wherein an increase in sCD28 levels in the subject indicates cancer relapse, thereby diagnosing cancer relapse.

By another aspect, there is provided a method of predicting cancer relapse in a subject in need thereof, the method comprising measuring sCD28 levels in the subject, wherein an increase in sCD28 levels in the subject is indicative of cancer relapse to come.

By another aspect, there is provided a method of determining response to PD-1/PD-L1 based immunotherapy in a subject in need thereof, the method comprising measuring sCD28 levels in the subject at at least 2 time points, wherein an increase in sCD28 levels indicates the subject is not responding or not a responder to the immunotherapy and a decrease or no change in sCD28 levels indicates the subject is responding or a responder to the immunotherapy, thereby determining response to PD-1/PD-L1 based immunotherapy.

In some embodiments, the CD28 is mammalian CD28. In some embodiments the CD28 is human CD28. In some embodiments, the human CD28 comprises or consists of the amino acid sequence:

(SEQ ID NO: 1)
MLRLLLALNLFPSIQVTGNKILVKQSPMLVAYDNA
VNLSCKYSYNLFSREFRASLHKGLDSAVEVCVVYG
NYSQQLQVYSKTGFNCDGKLGNESVTFYLQNLYVN
QTDIYFCKIEVMYPPPYLDNEKSNGTIIHVKGKHL
CPSPLFPGPSKPFWVLVVVGGVLACYSLLVTVAFI
IFWVRSKRSRLLHSDYMNMTPRRPGPTRKHYQPYA
PPRDFAAYRS

In some embodiments, mature CD28 lacks a signal peptide and comprises the sequence:

(SEQ ID NO: 2)
NKILVKQSPMLVAYDNAVNLSCKYSYNLFSREFRA
SLHKGLDSAVEVCVVYGNYSQQLQVYSKTGFNCDG
KLGNESVTFYLQNLYVNQTDIYFCKIEVMYPPPYL
DNEKSNGTIIHVKGKHLCPSPLFPGPSKPFWVLVV
VGGVLACYSLLVTVAFIIFWVRSKRSRLLHSDYMN
MTPRRPGPTRKHYQPYAPPRDFAAYRS.

In some embodiments, the DNA coding sequence that codes for full length human CD28 comprises the sequence:

(SEQ ID NO: 3)
ATGCTCAGGCTGCTCTTGGCTCTCAACTTATTCCC
TTCAATTCAAGTAACAGGAAACAAGATTTTGGTGA
AGCAGTCGCCCATGCTTGTAGCGTACGACAATGCG
GTCAACCTTAGCTGCAAGTATTCCTACAATCTCTT
CTCAAGGGAGTTCCGGGCATCCCTTCACAAAGGAC
TGGATAGTGCTGTGGAAGTCTGTGTTGTATATGGG
AATTACTCCCAGCAGCTTCAGGTTTACTCAAAAAC
GGGGTTCAACTGTGATGGGAAATTGGGCAATGAAT
CAGTGACATTCTACCTCCAGAATTTGTATGTTAAC
CAAACAGATATTTACTTCTGCAAAATTGAAGTTAT
GTATCCTCCTCCTTACCTAGACAATGAGAAGAGCA
ATGGAACCATTATCCATGTGAAAGGGAAACACCTT
TGTCCAAGTCCCCTATTTCCCGGACCTTCTAAGCC
CTTTTGGGTGCTGGTGGTGGTTGGTGGAGTCCTGG
CTTGCTATAGCTTGCTAGTAACAGTGGCCTTTATT
ATTTTCTGGGTGAGGAGTAAGAGGAGCAGGCTCCT
GCACAGTGACTACATGAACATGACTCCCCGCCGCC
CCGGGCCCACCCGCAAGCATTACCAGCCCTATGCC
CCACCACGCGACTTCGCAGCCTATCGCTCCTGA.

As used herein, sCD28 refers to any CD28 fragment or variant that does not comprise a transmembrane domain and thus cannot be integrated in a membrane. In some embodiments, the CD28 transmembrane domain comprises the amino acid sequence FWVLVVVGGVLACYSLLVTVAFIIFWV (SEQ ID NO: 4). In some embodiments, sCD28 is not membrane bound. In some embodiments, sCD28 is in solution. In some embodiments, the sCD28 is CD28 in blood. In some embodiments, the sCD28 is CD28 in the TME. In some embodiments, sCD28 is CD28 in a bodily fluid. In some embodiments, sCD28 is a cleavage product from membranal CD28 (mCD28). In some embodiments, sCD28 is truncated CD28. In some embodiments, sCD28 lacks the cytoplasmic domain of full-length CD28. In some embodiments, sCD28 is dimeric sCD28. In some embodiments, sCD28 is monomeric sCD28. In some embodiments, sCD28 is not a splice variant arising from alternative splicing of CD28. In some embodiments, sCD28 comprises the amino acid sequence: MLRLLLALNLFPSIQVTGNKILVKQSPMLVAYDNAVNLSCKYSYNLFSREFRASLHKG LDSAVEVCVVYGNYSQQLQVYSKTGFNCDGKLGNESVTFYLQNLYVNQTDIYFCKIE VMYPPPYLDNEKSNGTIIHVKGKHLCPSP (SEQ ID NO: 5). In some embodiments, sCD28 lacks the signal peptide and comprises the sequence:

(SEQ ID NO: 6)
NKILVKQSPMLVAYDNAVNLSCKYSYNLFSREFRA
SLHKGLDSAVEVCVVYGNYSQQLQVYSKTGFNCDG
KLGNESVTFYLQNLYVNQTDIYFCKIEVMYPPPYL
DNEKSNGTIIHVKGKHLCPSP.

In some embodiments, the subject previously had cancer. In some embodiments, the subject still has cancer. In some embodiments, the subject suffers from cancer. In some embodiments, the subject had cancer and is currently cancer free. In some embodiments, the subject has cancer that is in remission. In some embodiments, the subject has cancer that is in partial remission. In some embodiments, remission comprises complete remission and partial remission. In some embodiment remission comprises a partial response and a complete response. In some embodiments, remission comprises complete remission. In some embodiments, remission comprises partial remission. In some embodiments, the subject has undergone therapy for the cancer. In some embodiments, the subject is undergoing therapy for the cancer. In some embodiments, the therapy is immunotherapy. In some embodiments, the subject is a responder to the therapy. In some embodiments, the subject is a non-responder to the therapy. In some embodiments, the subject has undergone PD-1/PD-L1 based immunotherapy. In some embodiments, the subject is undergoing PD-1/PD-L1 based immunotherapy.

In some embodiments, a responder is a subject that is responsive to the therapy. In some embodiments, a responder is someone that is responding. In some embodiments, someone that is responding is a responder. In some embodiments, a responder is a subject with a favorable response to the therapy. As used herein, a “favorable response” of the cancer patient indicates “responsiveness” of the cancer patient to the treatment with the therapy, namely, the treatment of the responsive cancer patient with the immunotherapy will lead to the desired clinical outcome such as tumor regression, tumor shrinkage or tumor necrosis; an anti-tumor response by the immune system; preventing or delaying tumor recurrence, tumor growth or tumor metastasis. In some embodiments, response is complete response (CR). In some embodiments, response is partial response (PR). In some embodiments, the response is cancer regression. In some embodiments, regression is from stable disease. In some embodiments, regression is from progressive disease. In some embodiments, a non-responder is a subject that is not responsive to the therapy. In some embodiments, a non-responder is someone that is not responding. In some embodiments, someone that is not responding is a non-responder. In some embodiments, a non-responder is a subject with a non-favorable response to the therapy. As used herein a “non-favorable response” of the cancer patient indicates “non-responsiveness” of the cancer patient to the treatment with the therapy and thus the treatment of the non-responsive cancer patient with the therapy will not lead to the desired clinical outcome, and potentially to a non-desired outcome such as tumor expansion, recurrence and metastases. In some embodiments, the non-desired outcome is cancer relapse. In some embodiments, a non-desired response is development of stable disease. In some embodiments, a non-desired response is development of progressive disease.

As used herein, the terms “complete response”, “partial response”, “stable disease” and “progressive disease” are all evaluation criteria for assessing cancer and in particular solid lesions. During evaluation, lesions are measured in order to provide basis for comparison and evaluation during treatment. In some embodiments, complete response refers to all lesions having disappeared during the course of treatment. In some embodiments, complete response refers to all target lesions having disappeared during the course of treatment. In some embodiments, partial response refers to a decrease in the size of a lesion or lesions. In some embodiments, the decrease is size is of the largest lesion. In some embodiments, the decrease is in a sum of the sizes of all the lesions. In some embodiments, the decrease is in the average size of the lesions. In some embodiments, the decrease in size is a decrease of at least 10, 20, 30, 40, or 50%. Each possibility represents a separate embodiment of the invention. In some embodiments, the decrease is size is a decrease of at least 30%. In some embodiments, the decrease is a decrease in the diameter of the lesion. In some embodiments, stable disease refers to no significant decrease or increase in the size of a lesion or lesions. In some embodiments, progressive disease refers to an increase in the size of the lesion or lesions. In some embodiments, the increase is in the largest lesion. In some embodiments, the increase is in a sum of the sizes of all the lesions. In some embodiments, the increase is in the average size of the lesions. In some embodiments, the increase in size is an increase of at least 10, 20, 30, 40, or 50%. Each possibility represents a separate embodiment of the invention. In some embodiments, the increase is size is an increase of at least 20%.

In some embodiments, the immunotherapy is PD-1 and/or PD-L1 based immunotherapy. In some embodiments, the immunotherapy is PD-1 based immunotherapy. In some embodiments, the immunotherapy is PD-L1 based immunotherapy. In some embodiments, the PD-1/PD-L1 based immunotherapy comprises administering an anti-PD1 or anti-PD-L1 antibody. In some embodiments, the therapy comprises blockade of the PD-1 checkpoint. In some embodiments, the immunotherapy comprises administering allogenic, syngenic or autologous immune cells to the subject. In some embodiments, the immune cells are T cells. In some embodiments, the subject in need of immunotherapy suffers from cancer. In some embodiments, PD-1 based immunotherapy comprises PD-L2 based immunotherapy. In some embodiments, PD-L1 based immunotherapy comprises PD-L2 based immunotherapy.

In some embodiments, the immunotherapy is CD80 based immunotherapy. In some embodiments, the immunotherapy is CD86 based immunotherapy. CD80 and CD86 immunotherapies are well known in the art and comprise administering CD80/CD86 and or mimic, derivatives or mimetics thereof to stimulate an immune response. CD80-Fc is currently in clinical trials as an anticancer immunotherapeutic for non-limiting example.

In some embodiments, the immunotherapy is CTLA-4 based immunotherapy. In some embodiments, CTLA-4 immunotherapy comprises CTLA-4 blockade. In some embodiments, CTLA-4 immunotherapy comprises administering a CTLA-4 inhibitor. In some embodiments, CTLA-4 immunotherapy comprises administering an anti-CTLA-4 antibody. In some embodiments, the therapy comprises blockade of the CTLA-4 checkpoint. In some embodiments, the immunotherapy is a combination therapy of any of the herein described immunotherapies.

In some embodiments, the subject suffers from cancer. In some embodiments, the cancer is a cancer that can be treated with immunotherapy. In some embodiments, the cancer is a cancer that can be treated with PD-1/PD-L1 therapy. In some embodiments, the subject has undergone PD-1/PD-L1 therapy. In some embodiments, the subject is a non-responder to PD-1/PD-L1 therapy. In some embodiments, the subject is naïve to PD-1/PD-L1 therapy. In some embodiments, the methods of the invention are performed together with PD-1/PD-L1 therapy. In some embodiments, the methods of the invention are performed before PD-1/PD-L1 therapy.

In some embodiments, the cancer is a cancer with elevated sCD28 levels. In some embodiments, the cancer comprises high sCD28 levels. In some embodiments, elevated and/or high sCD28 levels are levels at and/or above 5, 6, 7, 8, 9, 10, 12, 14, 15, 17, 20, 25, 30, 35, 40, 50, 60, 70, 80, 90 or 100 ng/mL. Each possibility represents a separate embodiment of the invention. In some embodiments, the cancer comprises high sCD28 levels. In some embodiments, elevated and/or high sCD28 levels are levels at and/or above 5, 10, 15, 20, 25, 30, 35, 40, 45, 50, 55, 60, 65, 70, 75, 80, 85, 90, 95, 100, 150, 200, 300, 400, 500, 600, 700, 800, 900, or 1000% of the levels in a healthy subject. Each possibility represents a separate embodiment of the invention. In some embodiments, the cancer is not breast cancer. In some embodiments, the cancer is selected from melanoma, urothelial carcinoma, head and neck, non-small cell lung cancer, ovarian, kidney, gastric and colorectal. In some embodiments, the cancer is selected from melanoma, urothelial carcinoma, head and neck, non-small cell lung cancer, ovarian, and colorectal. In some embodiments, the cancer is melanoma, urothelial carcinoma, head and neck, non-small cell lung cancer, ovarian, kidney, gastric or colorectal. Each possibility represents a separate embodiment of the invention. In some embodiments, the cancer is selected from melanoma and urothelial carcinoma. In some embodiments, the cancer is selected from skin cancer, urothelial cancer, lung cancer, and renal cancer. In some embodiments, the cancer is selected from skin cancer and urothelial cancer.

In some embodiments, sCD28 is measured at at least 1, 2, 3, 4, 5, 6, 7, 8, 9 or 10 time points. Each possibility represents a separate embodiment of the invention. In some embodiments, sCD28 is measured at at least 2 time points. In some embodiments, the at least two time points comprise a first time point and a second time point. In some embodiments, there is an increase from one time point to a second time point. In some embodiments, there is a decrease from one time point to a second time point. In some embodiments, there is no change from one time point to a second time point. In some embodiments, there is a trend in the sCD28 kinetics. In some embodiments, the trend is an increase or a decrease. In some embodiments, an increasing trend indicates the subject is not responsive to therapy. In some embodiments, an increasing trend indicates cancer relapse. In some embodiments, an increasing trend indicates an imminent cancer relapse.

In some embodiments, a subject that responds to therapy is a responder. In some embodiments, a subject that does not respond to therapy is a non-responder. Response to therapy need not be a complete response but may be a partial response or a response of an improvement of at least one symptom. In some embodiments, a non-responder has no positive response to therapy.

In some embodiments, the increase is as compared to sCD28 levels in a healthy subject. In some embodiments, the increase is as compared to sCD28 levels in the subject before relapse. In some embodiments, the increase is as compared to sCD28 levels in the subject after initiation of the immunotherapy. In some embodiments, the increase is as compared to sCD28 levels after remission of the disease. In some embodiments, the increase is as compared to a predetermined threshold. In some embodiments, the increase is as compared to levels before initiation of immunotherapy.

In some embodiments, the increase is from one time point to another. In some embodiments, the increase is from a first time point to a second time point. In some embodiments, an increase indicates the subject is a non-responder to therapy. In some embodiments, an increase indicates the subject is not responding to therapy. In some embodiments, the method further comprises suspending therapy to a not responding patient. In some embodiments, the method further comprises increasing a dose to a not responding patient. In some embodiments, the increase is a statistically significant increase.

In some embodiments, at least one time point is at or before initiation of treatment. In some embodiments, at least one time point is after initiation of treatment. In some embodiments, the first time point is before therapy. In some embodiments, the first time point is at initiation of therapy. In some embodiments, the first time point is during remission. In some embodiments, the first time point is when the subject is cancer free. In some embodiments, the first time point is before initiation of immunotherapy. In some embodiments, the first time point is at initiation of immunotherapy. In some embodiments, the first time point is during immunotherapy. In some embodiments, the second time point is the current measuring. In some embodiments, the second time point is a test time point. In some embodiments, the second time point is a time point when the subject is at risk for relapse. In some embodiments, the second time point is after initiation of immunotherapy. In some embodiments, the second time point is during immunotherapy. In some embodiments, the first time point is before initiation of immunotherapy and the second time point is after initiation of immunotherapy. In some embodiments, the first and second time points are after initiation of immunotherapy.

In some embodiments, the first and second time points are separated by at least 1, 2, 3, 4, 5, 6, or 7 weeks. Each possibility represents a separate embodiment of the invention. In some embodiments, the first and second time points are separated by at least 6 weeks. In some embodiments, the first and second time points are separated by at least 7 weeks. In some embodiments, the first and second time points are separated by at most 1, 2, 3, 4, 5, 6, or 7 weeks. Each possibility represents a separate embodiment of the invention. In some embodiments, the first and second time points are separated by at most 6 weeks. In some embodiments, the first and second time points are separated by at most 7 weeks. In some embodiments, the first and second time points are separated by about 6 weeks. In some embodiments, the first and second time points are separated by about 7 weeks. In some embodiments, the first and second time points are separated by about 6-7 weeks. In some embodiments, the first and second time points are separated by 6-7 weeks. In some embodiments, the second time point is 7 weeks after initiation of immunotherapy. In some embodiments, the first time point is at initiation of immunotherapy. In some embodiments, the first time point is 7 weeks after initiation of immunotherapy. In some embodiments, the second time point is 13 weeks after initiation of immunotherapy. In some embodiments, the second time point is 7 weeks or 13 weeks after initiation of immunotherapy.

In some embodiments, the increase is at least a 10, 20, 25, 30, 40, 50, 60, 70, 80, 90, 100, 150, 200, 250, 300, 350, 400, 450, 500, 600, 700, 800, 900, or 1000% increase. Each possibility represents a separate embodiment of the invention. In some embodiments, the increase is an increase from no expression of sCD28 to expression. In some embodiments, the increase is from an absence of sCD28 to the presence of sCD28. In some embodiments, the increase is to at least 1, 2, 3, 4, 5, 6, 7, 8, 9 or 10 ng/mL sCD28. Each possibility represents a separate embodiment of the invention. In some embodiments, the increase is to at least 1 ng/mL sCD28. In some embodiments, the increase is to at least 2 ng/mL sCD28. In some embodiments, the increase is to at least 5 ng/mL sCD28. In some embodiments, the increase is to at least 6 ng/mL sCD28. In some embodiments, the increase is an increase of at least 1, 2, 3, 4, 5, 6, 7, 8, 9 or 10 ng/mL sCD28. Each possibility represents a separate embodiment of the invention. In some embodiments, the increase is an increase of at least 1 ng/mL sCD28. In some embodiments, the increase is an increase of at least 2 ng/mL sCD28.

In some embodiments, a decrease in sCD28 levels from a first time point to a second time point indicates the subject is a responder to the immunotherapy. In some embodiments, no change in sCD28 levels from a first time point to a second time point indicates the subject is a responder to the immunotherapy. In some embodiments, an increase in sCD28 levels from a first time point to a second time point indicates the subject is a non-responder to the immunotherapy. In some embodiments, the decrease is from one time point to another. In some embodiments, the decrease is from a first time point to a second time point. In some embodiments, a decrease indicates the subject is a responder to therapy. In some embodiments, a decrease indicates the subject is responding to therapy. In some embodiments, the method further comprises continuing therapy to a responding patient. In some embodiments, the method further comprises increasing therapy to a responding patient. In some embodiments, the decrease is a statistically significant decrease. In some embodiments, the method comprises discontinuing therapy to a non-responding patient. In some embodiments, the method comprises administering a different therapy to a non-responding patient. In some embodiments, a different therapy is another therapy.

In some embodiments, the no change is from one time point to another. In some embodiments, the no change is from a first time point to a second time point. In some embodiments, no change indicates the subject is a responder to therapy. In some embodiments, no change indicates the subject is responding to therapy. In some embodiments, no change comprises a statistically insignificant change. In some embodiments, the decrease is at least a 5, 10, 15, 20, 25, 30, 35, 40, 45, 50, 55, 60, 65, 70, 75, 80, 85, 90, 95, 97, 99 or 100% decrease. Each possibility represents a separate embodiment of the invention. In some embodiments, the decrease is a decrease to no expression of sCD28. In some embodiments, the decrease is a decrease to substantially no expression of sCD28. In some embodiments, the decrease is to less than 1, 2, 3, 4, 5, 6, 7, 8, 9 or 10 ng/mL sCD28. Each possibility represents a separate embodiment of the invention. In some embodiments, the decrease is to less than 2 ng/mL sCD28. In some embodiments, the decrease is to less than 5 ng/mL sCD28. In some embodiments, the decrease is to less than 6 ng/mL sCD28. In some embodiments, the decrease is a decrease of at least 1, 2, 3, 4, or 5 ng/mL sCD28. Each possibility represents a separate embodiment of the invention. In some embodiments, the decrease is a decrease of at least 1 ng/mL sCD28. In some embodiments, the decrease is a decrease of at least 2 ng/mL sCD28.

In some embodiments, the subject's blood comprises elevated levels of sCD28. In some embodiments, the increase is to elevated levels. In some embodiments, the levels are elevated above those of healthy subjects. In some embodiments, the subject's sCD28 levels are elevated by at least 5%, 10%, 15%, 20%, 25%, 30%, 40%, 50%, 60%, 70%, 80%, 90%, 100%, 150%, 200%, 250%, 300%, 350%, 400%, 500%, 600%, 700%, 800%, 900%, or 1000% above healthy subject levels. Each possibility represents a separate embodiment of the invention. In some embodiments, the levels are elevated above 5, 6, 7, 8, 9, 10, 12, 14, 15, 16, 18, 20, 25, 30, 35, 40, 45 or 50 ng/mL of blood. Each possibility represents a separate embodiment of the invention. In some embodiments, the levels are elevated above 5 ng/mL. In some embodiments, the levels are elevated above 10 ng/mL. In some embodiments, the levels are elevated above 20 ng/mL. In some embodiments, the subject's blood comprises at least 5, 6, 7, 8, 9, 10, 12, 14, 15, 16, 18, 20, 25, 30, 35, 40, 45 or 50 ng sCD28 per mL of blood. Each possibility represents a separate embodiment of the invention. In some embodiments, the subject's blood comprises at least 5 ng/mL sCD28. In some embodiments, the subject's blood comprises at least 10 ng/mL sCD28. In some embodiments, the subject's blood comprises at least 20 ng/mL sCD28.

In some embodiments, the subject's blood comprises healthy levels of sCD28 before relapse. In some embodiments, the subject's comprises non-elevated levels of sCD28 before relapse. In some embodiments, the subject's blood comprises less than 1, 2, 3, 4, 5, 6, 7, 8, 9, 10, 12, 14, 15, 16, 18, 20, 25, 30, 35, 40, 45 or 50 ng/mL sCD28 before relapse. In some embodiments, the subject's blood comprises less than 5 ng/mL sCD28 before relapse. In some embodiments, the subject's blood comprises less than 6 ng/mL sCD28 before relapse. Each possibility represents a separate embodiment of the invention. In some embodiments, the subject's blood is the subject's blood before performance of a method of the invention.

In some embodiments, the measuring comprises obtaining a sample from the subject. In some embodiments, the measuring is measuring sCD28 levels in the sample. In some embodiments, the sample is a biopsy. In some embodiments, the sample is a bodily fluid. In some embodiments, the sample is blood. In some embodiments, the sample is from a subject. In some embodiments, the sample comprises a bodily fluid. In some embodiments, the sample comprises tissue. In some embodiments, the sample comprises cells. In some embodiments, the detection is by a secondary antibody. In some embodiments, the detection is with a tagged molecule that binds the sCD28. In some embodiments, the detection is by ELISA. In some embodiments, the detection is by immunohistochemistry. In some embodiments, the detection is by immunoblot. Agents for detecting sCD28 are described in International Patent Application WO2019/175885, herein incorporated by reference in its entirety.

In some embodiments, measuring comprises measuring at a plurality of time points. In some embodiments, the plurality of time points is at least 2, 3, 4, 5, 6, 7, 8, 9, or 10 time points. Each possibility represents a separate embodiment of the invention. In some embodiments, the plurality of time points is two time point. In some embodiments, the plurality of time points is at least 2 time points. In some embodiments, the plurality of time points comprise a first and a second time point. In some embodiments, at least one time point is at a time when the subject is in remission or cancer free. In some embodiments, at least one time point is at a time when the subject is known to be in remission or cancer free. In some embodiments, a time point when the subject is in remission or cancer free or known to be in remission or cancer free is a time point when the subject has sCD28 levels at or below 1, 2, 3, 4, 5, 6, 7, 8, 9 or 10 ng/mL sCD28. Each possibility represents a separate embodiment of the invention. In some embodiments, the time point is when the subject has sCD28 levels at or below 2 ng/mL. In some embodiments, the time point is when the subject has sCD28 levels at or below 5 ng/mL. In some embodiments, the time point is when the subject has sCD28 levels at or below 6 ng/mL. In some embodiments, at least one time point is after initiation of therapy.

In some embodiments, at least one time point is at a time when the subject is at risk of relapse. In some embodiments, at least one time point is at a time when the subject is suspected of relapse. In some embodiments, at least one time point is at least 1, 2, 3, 4, 5, 6, 7, 8, 9, 10, 15, 20, 25, 30, 35, 40, 45, 50, 55 or 60 weeks after initiation of therapy. Each possibility represents a separate embodiment of the invention. In some embodiments, at least one time point is at least 35 weeks after initiation of therapy. In some embodiments, at least one time point is at least 40 weeks after initiation of therapy. In some embodiments, at least one time point is at least 45 weeks after initiation of therapy. In some embodiments, at least one time point is at least 46 weeks after initiation of therapy.

In some embodiments, a relapse to come is an impending relapse. In some embodiments, impending relapse is imminent relapse. In some embodiments, impending is within the next 10, 15, 20, 25, 30, 35, or 40 weeks. Each possibility represents a separate embodiment of the invention. In some embodiments, a relapse to come is relapse at any time in the future. In some embodiments, imminent relapse is within the next 1, 2, 3, 4, 5, 6, 7, 8, 9, 10, 15 or 20 weeks. Each possibility represents a separate embodiment of the invention.

In some embodiments, the method further comprises administering another therapy to the subject. In some embodiments, the another therapy is a different therapy. In some embodiments, the another therapy is a therapy that treats the cancer. In some embodiments, the method further comprises administering another therapy to a subject diagnosed with or predicted for a cancer relapse. In some embodiments, the method further comprises administering another therapy to a subject diagnosed with a cancer relapse. In some embodiments, the method further comprises administering another therapy to a subject predicted for a cancer relapse. In some embodiments, the method further comprises administering another therapy to a subject determined to be a non-responder. In some embodiments, the method further comprises administering another therapy to a subject determined to be a non-responder to the immunotherapy. In some embodiments, the other therapy is not the therapy that caused the remission. In some embodiments, the other therapy is a different therapy than the therapy initially used to treat the cancer. In some embodiments, the other therapy is not a PD-1/PD-L1 based immunotherapy. In some embodiments, the other therapy is not an immunotherapy. In some embodiments, the other therapy is not the therapy to which the subject is a non-responder.

In some embodiments, the another therapy is an immunotherapy. In some embodiments, the another therapy is a therapy that treats the cancer. In some embodiments, the another therapy is a PD-1 and/or PD-L1 based immunotherapy. In some embodiments, the another immunotherapy is a checkpoint inhibitor. In some embodiments, the checkpoint inhibitor is a PD-1 and/or PD-L1 inhibitor. In some embodiments, the checkpoint inhibitor is a CTLA-4 inhibitor. In some embodiments, the another immunotherapy is a chimeric antigen receptor (CAR) based immunotherapy. In some embodiments, the CAR is a CAR-T. In some embodiments, the CAR is a CAR-NK. In some embodiments, the another immunotherapy is a cancer vaccine.

As used herein, the terms “administering,” “administration,” and like terms refer to any method which, in sound medical practice, delivers a composition containing an active agent to a subject in such a manner as to provide a therapeutic effect. One aspect of the present subject matter provides for oral administration of a therapeutically effective amount of an agent of the invention to a patient in need thereof. Other suitable routes of administration can include parenteral, subcutaneous, intravenous, intramuscular, or intraperitoneal.

As used herein, the terms “CAR-T cell” and “CAR-NK cell” refer to an engineered receptor which has specificity for at least one protein of interest (for example an immunogenic protein with increased expression following treatment with an epigenetic modifying agent) and is grafted onto an immune effector cell (a T cell or NK cell). In some embodiments, the CAR-T cell has the specificity of a monoclonal antibody grafted onto a T-cell. In some embodiments, the CAR-NK cell has the specificity of a monoclonal antibody grafted onto a NK-cell. In some embodiments, the T cell is selected from a cytotoxic T lymphocyte and a regulatory T cell.

CAR-T and CAR-NK cells and their vectors are well known in the art. Such cells target and are cytotoxic to the protein for which the receptor binds. In some embodiments, a CAR-T or CAR-NK cell targets at least one viral protein. In some embodiments, a CAR-T or CAR-NK cell targets a plurality of viral proteins. In some embodiments, a CAR-T or CAR-NK cell targets a viral protein with increased expression due to contact with an epigenetic modifying agent.

Construction of CAR-T cells is well known in the art. In one non-limiting example, a monoclonal antibody to a viral protein can be made and then a vector coding for the antibody will be constructed. The vector will also comprise a costimulatory signal region. In some embodiments, the costimulatory signal region comprises the intracellular domain of a known T cell or NK cell stimulatory molecule. In some embodiments, the intracellular domain is selected from at least one of the following: CD3Z, CD27, CD28, 4-1BB, OX40, CD30, CD40, PD-1, ICOS, lymphocyte function-associated antigen-1 (LFA-1) CD2, CD 7, LIGHT, NKG2C, B7-H3, and a ligand that specifically binds with CD83. In some embodiments, the vector also comprises a CD3Z signaling domain. This vector is then transfected, for example by lentiviral infection, into a T-cell.

As used herein, the terms “treatment” or “treating” of a disease, disorder, or condition encompasses alleviation of at least one symptom thereof, a reduction in the severity thereof, or inhibition of the progression thereof. Treatment need not mean that the disease, disorder, or condition is totally cured. To be an effective treatment, a useful composition herein needs only to reduce the severity of a disease, disorder, or condition, reduce the severity of symptoms associated therewith, or provide improvement to a patient or subject's quality of life.

As used herein, the term “relapse” refers to a worsening of the cancer after a period of improvement. Relapse need not mean that the cancer has returned to the level before treatment, but only that it has worsened in some way from the time it was in remission. In some embodiments, relapse comprises a return of the cancer after the subject was cancer free. In some embodiments, relapse comprises continued growth of the cancer. In some embodiments, relapse comprises growth of the cancer after a stoppage of growth. In some embodiments, relapse comprises an increase in the rate of growth of the cancer. In some embodiments, relapse comprises return of a symptom of the cancer than had stopped. In some embodiments, relapse comprises worsening of a symptom of the cancer than had been improved. Relapse need only be an increase in the severity of the cancer, or severity of symptoms associated with the cancer.

In some embodiments, the relapse is the relapsed cancer. In some embodiments, the relapse is refractive to the therapy that caused the remission. In some embodiments, the relapse is non-responsive to the therapy that caused the remission.

In some embodiments, the method is performed in vivo. In some embodiments, the method is performed ex vivo. In some embodiments, the method is performed in vitro. In some embodiments, the measuring is performed in vivo. In some embodiments, the measuring is performed ex vivo. In some embodiments, the measuring is performed in vitro. In some embodiments, the sample is an in vitro sample. In some embodiments, the sample is an ex vivo sample.

As used herein, the term “about” when combined with a value refers to plus and minus 10% of the reference value. For example, a length of about 1000 nanometers (nm) refers to a length of 1000 nm+−100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Strategies for Protein Purification and Characterization—A Laboratory Course Manual” CSHL Press (1996); all of which are incorporated by reference. Other general references are provided throughout this document.

### Materials and Methods

Detection of soluble human CD28 in cancer patients' plasma—Cancer patients plasma samples of different indications, that received anti-PD1 therapy, were diluted 1:10 and analyzed for soluble human CD28 by functional ELISA. Samples with high sCD28 were analyzed again in adequate dilutions. Samples were examined only if clinical observation on response to treatment was available. Examples were chosen by criteria of >0.5-fold change in amount of sCD28 during treatment only.

### Example 1: sCD28 Levels Before Immunotherapy are Predictive of Response

The immunosuppressive effect of sCD28 has already been established (International Patent Application WO2019/175885, herein incorporated by reference in its entirety). Further, it was found that sCD28 is a potent inhibitor of immunotherapy, and that levels of sCD28 are elevated in a number of cancers. Anti-PD1/anti-PD-L1 and CD80 based immunotherapies were all shown to be negatively impacted by sCD28.

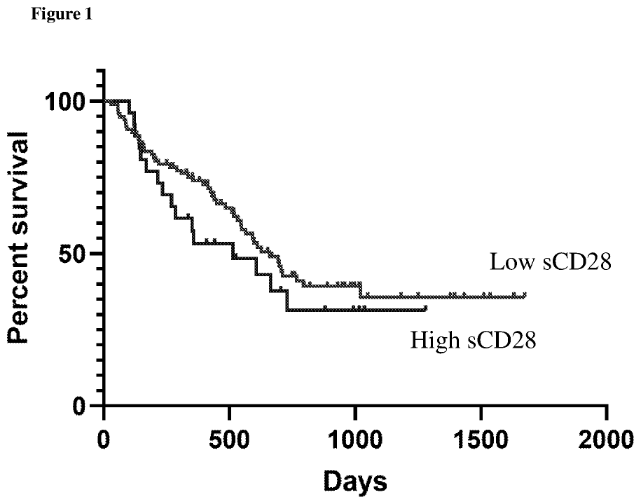

To further understand the impact of sCD28 levels on cancer progression during immunotherapy, more than 100 serum samples from melanoma patients prior to initiation of anti-PD1 therapy (Nivolumab, NCT01176461) were tested for sCD28 levels by ELISA. Survival was monitored throughout therapy and a survival function was plotted comparing subjects with high and low levels of sCD28 before therapy (FIG. 1). It was found that subjects with low levels of sCD28 (below 2 ng/mL) had a longer median survival than those with high levels (659 days vs. 514 days).

### Example 2: sCD28 Levels During Immunotherapy

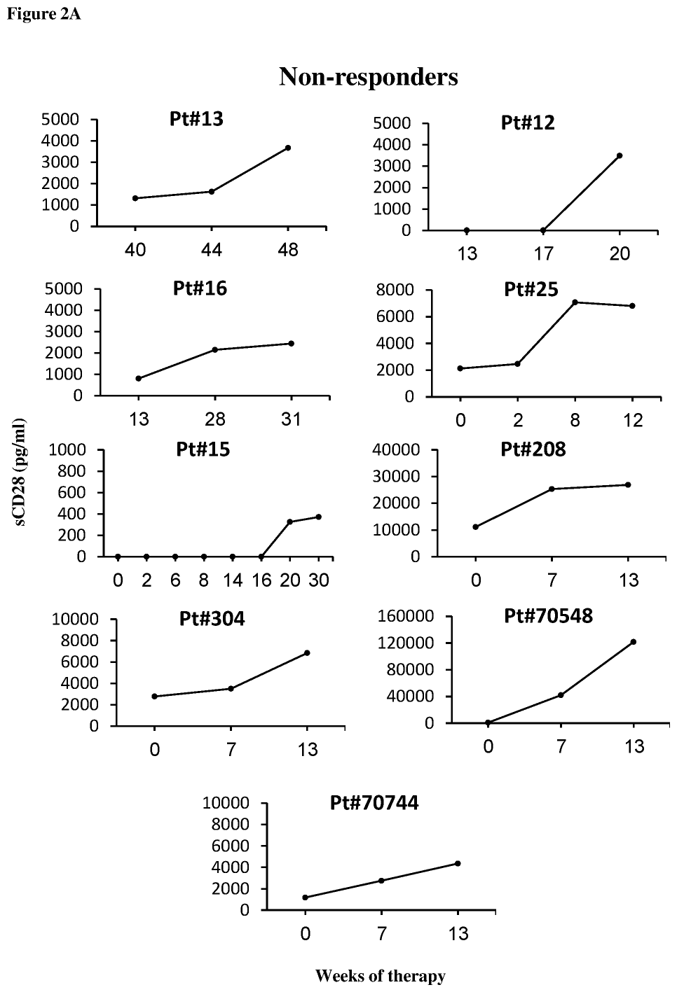

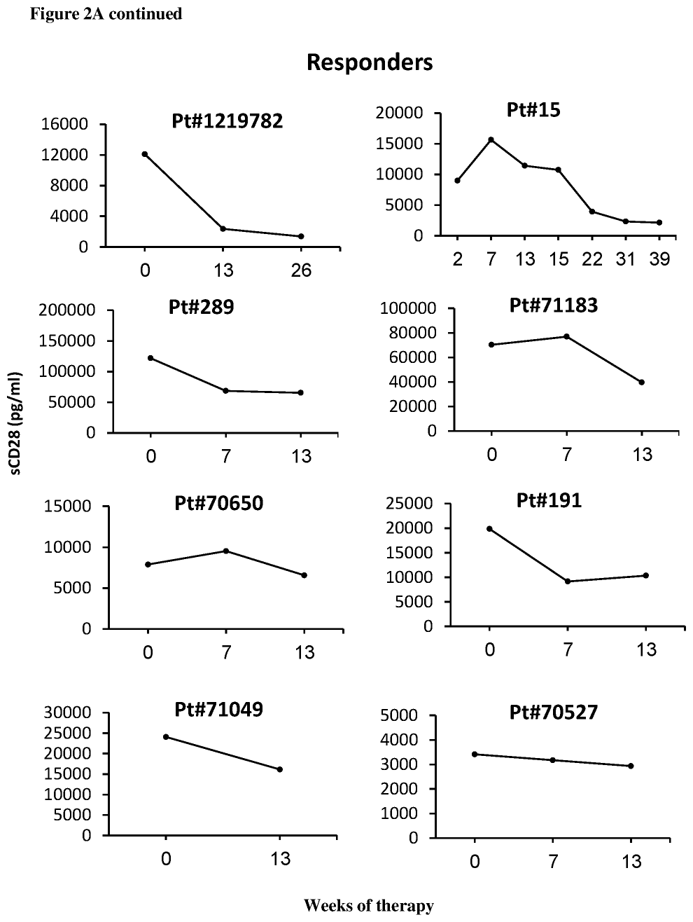

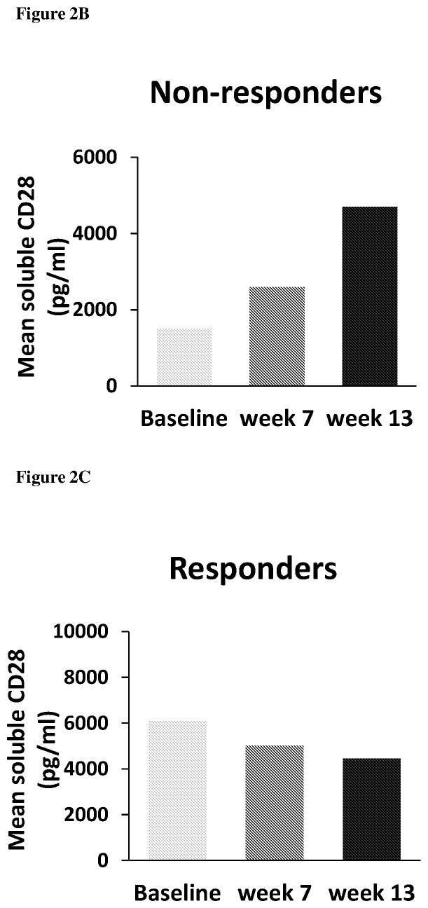

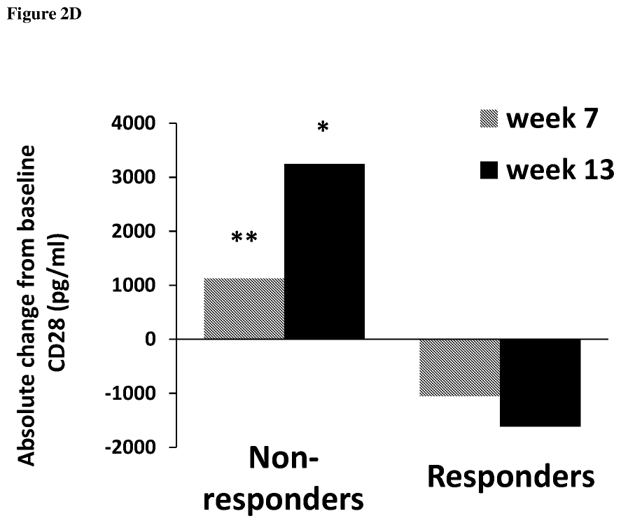

Serum from 166 individual patients (melanoma, renal cell carcinoma, lung squamous cell carcinoma and urothelial carcinoma patient) undergoing immunotherapy (anti-PD-1, for example Nivolumab and Pembrolizumab, anti-PD-L1, for example Atezolizumab and anti-CTLA-4, for example Ipilimumab) were examined for their sCD28 levels. Of these 166, 154 individuals provided multiple samples during the course of therapy. 37 of these subjects were considered to be positive for sCD28 (>2 ng/mL) for at least one time point measured. Of those 37, 19 showed altering kinetics of sCD28 occurrence during therapy (a greater than 50% change in sCD28 levels). Interestingly, the direction of change (positive of negative kinetics) was always consistent: responders had a decrease in sCD28 levels and non-responders showed an increase in sCD28 levels (FIG. 2A).

From these 166 patients, 92 individual patients were undergoing anti-PD-1 therapy with Nivolumab (NCT01176461). All the patients provided samples at three time point (baseline, week 7 and week 13) throughout the course of the immunotherapy. Patients were stratified into responders and non-responders based on cancer remission, as measured by tumor size, at the end of the trial. When the trend in sCD28 levels was examined in the responder and non-responder populations as a whole, the implications of sCD28 as a prognostic marker are readily apparent. Non-responders showed increasing sCD28 levels from baseline (before therapy) to week 7 and through to week 13 (FIG. 2B). In contrast, responders not only did not show increasing levels of sCD28, but in fact the average sCD28 levels decreased from baseline to week 7 and through to week 13 (FIG. 2C). When the absolute change in sCD28 expression is quantified at weeks 7 and 13 the increases seen in the non-responder population were statistically significant (FIG. 2D).

### Example 3: sCD28 Levels During Relapse

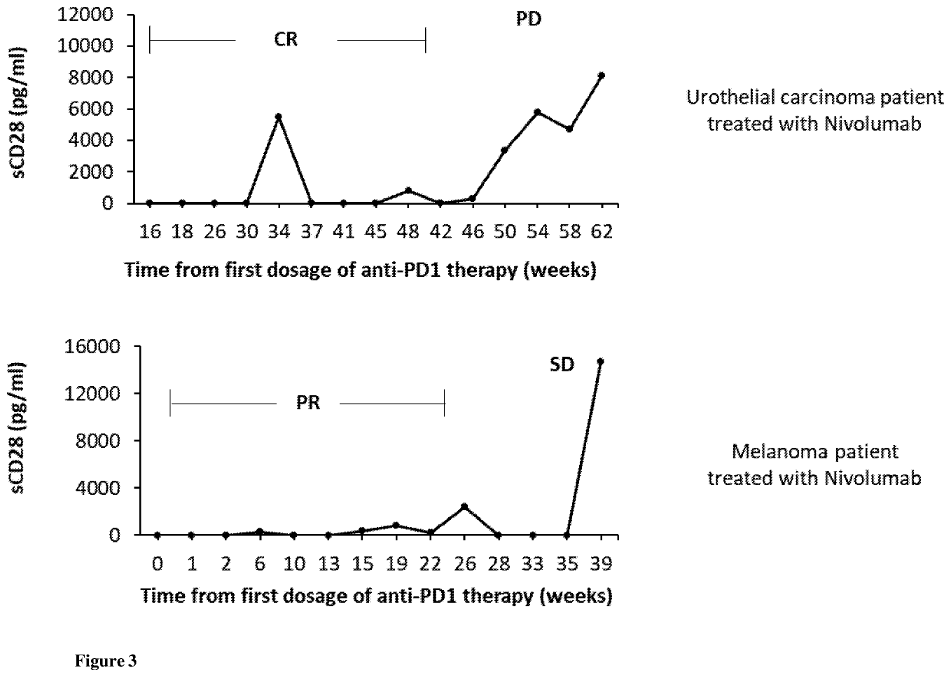

Two of the monitored patients that showed an alteration in kinetics of sCD28 showed an unexpected result. Both patients responded to immunotherapy, with the first (urothelial carcinoma) showing complete response (CR) and the other (melanoma) showing partial response (PR). Both patients were, on average, negative for sCD28 during their response to the therapy, that is during the periods of remission (FIG. 3). However, both patients showed a marked increase in sCD28 levels just before or at the initiation of cancer relapse. The patient that had a complete response entered a phase of progressive disease (PD) and sCD28 levels began increasing from week 46 and on (FIG. 3, upper panel). The patient that had a partial response entered a phase of stable disease (SD) and sCD28 levels began increasing at week 35 (FIG. 3, lower panel). This demonstrates that increased and/or increasing sCD28 levels can act as a marker for cancer relapse after immunotherapy, and that this increase can be a predictive marker for imminent relapse.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A method of determining response to PD-1/PD-L1 based immunotherapy in a subject suffering from cancer, the method comprising measuring sCD28 levels in said subject at at least two time points wherein at least one of those time points is after initiation of said PD-1/PD-L1 based immunotherapy, wherein an increase in sCD28 levels from a first time point to a second time point after initiation of said PD-1/PD-L1 based immunotherapy indicates said subject is not a responder to said immunotherapy and a decrease or no change in sCD28 levels from said first time point to said second time point after initiation of said PD-1/PD-L1 based immunotherapy indicates said subject is a responder to said immunotherapy, optionally wherein a decrease is a decrease of at least 1 ng/ml sCD28, thereby determining response to PD-1/PD-L1 based immunotherapy in a subject.

2. The method of claim 1, wherein said first time point is before or at the time of initiation of said immunotherapy.

3. The method of claim 1, wherein said first time point is after initiation of said immunotherapy.

4. The method of claim 1, wherein said second time point is at least 6 weeks after said first time point, at most 7 weeks after said first time point or both.

5. (canceled)

6. The method of claim 1, wherein
a. said first time point is at or before initiation of said immunotherapy and said second time point is 7 weeks or 13 weeks after initiation of said immunotherapy; or
b. said first time point is at 7 weeks after initiation of said immunotherapy and said second time point is 13 weeks after initiation of said immunotherapy.

7. The method of claim 1, wherein a decrease in sCD28 levels from said first time point to said second time point after initiation of said PD-1/PD-L1 based immunotherapy indicates said subject is a responder to said immunotherapy.

8. (canceled)

9. The method of claim 1, further comprising at least one of:
a. discontinuing said immunotherapy to a subject that is not a responder;
b. administering a different immunotherapy to a subject that is a non-responder;
c. continuing to administer said PD-1/PD-L1 based immunotherapy to a subject that is a responder; and
d. increasing a dose of said PD-1/PD-L1 based immunotherapy administered to a subject that is a responder.

10. A method of diagnosing or predicting cancer relapse in a subject in need thereof, the method comprising measuring soluble CD28 (sCD28) levels in said subject, wherein an increase in sCD28 levels in said subject indicates cancer relapse or an imminent cancer relapse, thereby diagnosing or predicting cancer relapse in said subject.

11. (canceled)

12. The method of claim 10, wherein said subject:
a. suffers from cancer and said cancer is in remission
b. previously had cancer and is currently cancer free;
c. has undergone or is undergoing immunotherapy, optionally wherein said immunotherapy is PD-1, PD-L1 and/or CD80 based immunotherapy; or
d. a combination thereof.

13. (canceled)

14. (canceled)

15. (canceled)

16. The method of claim 10, wherein said increase is:
a. as compared to sCD28 levels in said subject before relapse;
b. as compared to a predetermined threshold;
c. an increase of at least 50%;
d. an increase to at least 6 ng/mL sCD28;
e. an increase of at least 1 ng/ml sCD28; or
f. a combination thereof.

17. (canceled)

18. (canceled)

19. (canceled)

20. (canceled)

21. The method of claim 1, wherein said measuring comprises obtaining a sample from said subject and measuring sCD28 levels in said sample, optionally wherein said sample is a blood sample.

22. (canceled)

23. The method of claim 10, wherein said measuring comprises measuring at a plurality of time points, wherein at least one time point is at a time when said subject is known to be in remission or cancer free, and wherein at least one time point is at a time when the subject is at risk of relapse.

24. The method of claim 23, wherein remission comprises a partial response and a complete response to a therapy.

25. The method of claim 23, wherein at said time point when said subject is known to be in remission or cancer free sCD28 levels are below 5 ng/mL.

26. The method of claim 10, wherein said subject before performance of said method has blood sCD28 levels below 6 ng/mL.

27. The method of claim 1, wherein said cancer is selected from skin cancer, urothelial cancer, lung cancer, and renal cancer.

28. The method of claim 27, wherein said cancer is selected from melanoma, and urothelial cancer.

29. The method of claim 10, wherein imminent is within the next 20 weeks.

30. The method of claim 1, further comprising administering another immunotherapy to a subject diagnosed with or predicted for cancer relapse or determined to be a non-responder to said immunotherapy.

31. The method of claim 30, wherein said immunotherapy is selected from:
a. a checkpoint inhibitor, optionally wherein said checkpoint inhibitor is a PD-1 and/or PD-L1 based immunotherapy;
b. a chimeric antigen receptor (CAR) based therapy; and
c. a cancer vaccine.

32. (canceled)
